(19) United States
(12) Patent Application Publication
(10) Pub. No.: US 2011/0144384 A1
(43) Pub. Date: Jun. 16, 2011

(54) LIQUID-GAS PHASE REACTOR SYSTEM

(76) Inventors: Luciano Piras, Opera, MI (US); Marzio Monagheddu, Verona (IT); Andrea Gnagnetti, Monza (IT); Kishore K. Kar, Midland, MI (US)

(21) Appl. No.: 12/442,117

(22) PCT Filed: Sep. 20, 2007
(86) PCT No.: PCT/US07/20399

§ 371 Date: Sep. 9, 2010

Related U.S. Application Data
(60) Provisional application No. 60/846,783, filed on Sep. 22, 2006.

(51) Int. Cl. C07C 51/265; B01J 7/02; B01J 19/00

(52) U.S. Cl. 562/414; 422/236; 422/224

(57) ABSTRACT

A liquid-gas phase reactor system including a slinger located in an upper section (headspace region) of a reaction vessel. The slinger comprises an upper horizontal surface including a plurality of vertically raised vanes extending radially outward along a curved path which effectively distribute liquid about the reactor vessel. A method for conducting an oxidation reaction using a liquid-gas phase reactor system is also disclosed. The disclosed reactor system and method have a broad range of applications but are particularly suited for the production of terephthalic acid.

### CROSS-REFERENCE TO RELATED APPLICATION

This application claims the benefit of U.S. Provisional Application No. 60/846,783, filed Sep. 22, 2006.

### BACKGROUND OF THE INVENTION

(1) Field of the Invention

The invention relates to liquid-gas phase reactor systems and methods for conducting liquid-gas phase reactions. Such reactions include both liquid and gas phase constituents within the same reaction vessel, such as the oxidation of aromatic alkyls (e.g. p-xylene) within a liquid phase reaction medium.

(2) Description of the Related Art

Liquid-gas phase reactor systems are well known in the art and typically comprise a reaction vessel with optional auxiliary equipment. Reaction vessels including agitation devices are sometimes also referred to as “stirred tank reactors” or simply “STR” and those including oxygen-containing gas spargers as “liquid oxidation reactors” or “LOR” (see for example U.S. Pat. Nos. 5,108,662 and 5,536,875). Such reactor systems are commonly used in fermentations, hydrogenations, phosgenation, neutralization, chlorinations and oxidation reactions where it is necessary to make intimate' contact between liquid and gas phase constituents. To improve mass transfer between liquid and gas phase constituents, agitation devices are often included within the reaction vessel. For example, WO 01/41919 published Jun. 14, 2001 to K. Kar and L. Piras describes a liquid-gas phase reactor system including an agitation system comprising a draft tube and a combination of axial and radial impellers for improving mixing of gas and liquid phase constituents. Similarly, U.S. Pat. No. 6,984,753 which issued Jan. 10, 2006 to A. Gnagnetti, K. Kar and L. Piras describes a liquid-gas phase reactor system for oxidizing dimethylbenzenes within a reaction vessel equipped with an agitation device including a gas dispersing radial impeller having multiple parabolic shaped blades (e.g. Bakker Turbine BT6 model) in combination with an axial impeller (e.g. pitch blade turbine) operating in down pumping mode where oxygen-containing gas is sparged through nozzles near the tips of the axial impeller. In one embodiment, air is sparged through a liquid phase reaction medium of p-xylene, acetic acid, catalyst (i.e. cobalt and manganese) and initiator (bromide ion). Heat generated by the exothermic oxidation reaction is dissipated by the vaporization of solvent and water produced by the oxidation of p-xylene (i.e. “reaction water”). The temperature in the reaction vessel is controlled by the vaporization of solvent and reaction water and by the recycle of the condensate stream of the overhead vapors. The reaction conditions within the vessel are normally maintained at approximately 180-205° C. and at a pressure of approximately 14-18 bar. Crude terephthalic acid is recovered from the reaction product effluent via crystallization and filtration.

U.S. Pat. No. 5,102,630 to Lee describes a similar reactor system and oxidation reaction wherein vaporized solvent and reaction water pass upwardly out of the reactor to an overhead condenser system where at least a portion of the vapor is condensed and returned to the reaction vessel via a conduit from the top of the vessel. U.S. Pat. No. 5,099,064 to Huber et al. discloses a similar process wherein a condenser is combined with a separating system for separating out solvent-rich portions from the condensate which are then combined with fresh liquid feed steam and re-introduced into the lower side or bottom of the vessel at a location below the liquid level within the vessel. Similarly, U.S. Pat. No. 6,949,673 to Housley et al. describes a modified system wherein condensate may be returned to the reaction vessel headspace via an efflux slinger and/or to the liquid phase reaction medium at a location below the liquid level in the vessel via a separate feed line or by mixing with the existing feed stream.

Many liquid-gas phase chemical reactions generate solid phase reaction products. For example, the catalyzed oxidation of p-xylene within acetic acid can produce crystals of terephthalic acid. In industrial scale reactor systems, most of the terephthalic acid crystals remain suspended within the liquid phase. However, crystals can build-up on the walls of the reaction vessel (“wall fouling”) and can be entrained along with other solid debris in rising vapor which can lead to plugging of the condenser inlets (“condenser plugging”). Many of these problems are described in US 2004/0234435 published Nov. 25, 2004.

The use of a slinger to distribute condensate back to the reaction vessel can reduce both wall fouling and condenser plugging; however, conventional slinger designs provide only a modest improvement. For example, a conventional slinger used in such applications comprises a rotating, flat circular disk with a plurality of vertically raised, straight vanes extending radially outward from a center hub of the disk to its outer periphery. The slinger is located in the upper “head space” section of the vessel. Condensate is returned to the vessel via a conduit located above the rotating slinger. Condensate is fed onto the slinger where it is subsequently “slung” or distributed radially outward about the vessel. One shortcoming of this slinger is that the majority of condensate is distributed only over a limited cross-section of vessel with little condensate actually reaching the reactor walls. A second shortcoming is that liquid tends to be distributed in large droplets rather than finely divided droplets. Consequently, such systems experience wall fouling, condenser plugging, and poor mixing of condensate with the liquid phase reaction medium. Moreover, the present inventors have found that the aforementioned slinger is less effective at dissipating heat generated by exothermic reactions as compared with returning condensate to the vessel via a liquid inlet at a location below the liquid level, (e.g. with incoming fresh liquid reaction medium). For example, with the exothermic oxidation of aromatic alkyls, much of the heat generated by the reaction is concentrated in the middle section of the liquid reaction medium. These “hot spots” can lead to undesired reactions, consumption of solvent and increased vapor generation—all of which contribute to higher operating costs and lower efficiency. Additional studies by the present inventors have also demonstrated that the use of such a slinger provides less effective mixing of condensate with the liquid phase reaction medium, as compared with returning condensate via a liquid feed line at a point below the liquid level in the vessel—such as with the feed line used for introducing fresh liquid reaction medium.

The slingers described above are associated with the distribution of liquids as used in liquid-gas phase reactor systems. Slingers are also used in non-analogous arts, such as those involving the mixing of sand and other solids, see for example U.S. Pat. Nos. 4,453,829 and 4,808,004.

### BRIEF SUMMARY OF THE INVENTION

One embodiment of the subject invention is a liquid-gas phase reactor system including a reaction vessel, a liquid inlet and a slinger. The slinger comprises an upper horizontal surface including a plurality of vertically raised vanes extending radially outward along a curved path which effectively distributes liquid (e.g. fresh feed, condensate, etc.) to the reaction vessel. In yet another embodiment, the invention is a method for oxidizing an organic reactant within a liquid-gas phase reactor system. Other embodiments are also disclosed. While the invention finds broad utility in performing reactions involving both gas and liquid phases, e.g. fermentations, hydrogenations, phosgenation, neutralization, and chlorinations; the invention finds particular utility in the oxidation of aromatic alkyls such as p-xylene.

### BRIEF DESCRIPTION OF THE DRAWINGS

FIG. 1 is a schematic diagram of one embodiment of a liquid-gas reactor system.

FIG. 2 is a perspective view of one embodiment of the subject slinger.

FIG. 3 is a perspective view of another embodiment of the subject slinger.

### DETAILED DESCRIPTION OF THE INVENTION

The present invention includes a liquid-gas phase reactor system and a method for oxidizing an organic reactant within a liquid-gas phase reactor system. The reactor system includes a reaction vessel, also referred herein as simply “vessel” or “reactor”. The vessel itself is not particularly critical to the invention and may comprise many boiling-type reactor configurations. As with most reaction systems, the nature of the chemical process will dictate the configuration and construction materials of the vessel and auxiliary equipment. For example, stainless steel or titanium materials are often used with highly corrosive chemical processes whereas carbon-based steels may be applicable for non-corrosive environments. For most applications, the vessel includes a circular cross-section such as a vertically aligned cylinder with an upper section corresponding to the head space region and a lower section corresponding to the liquid level of the liquid phase reaction medium within the vessel.

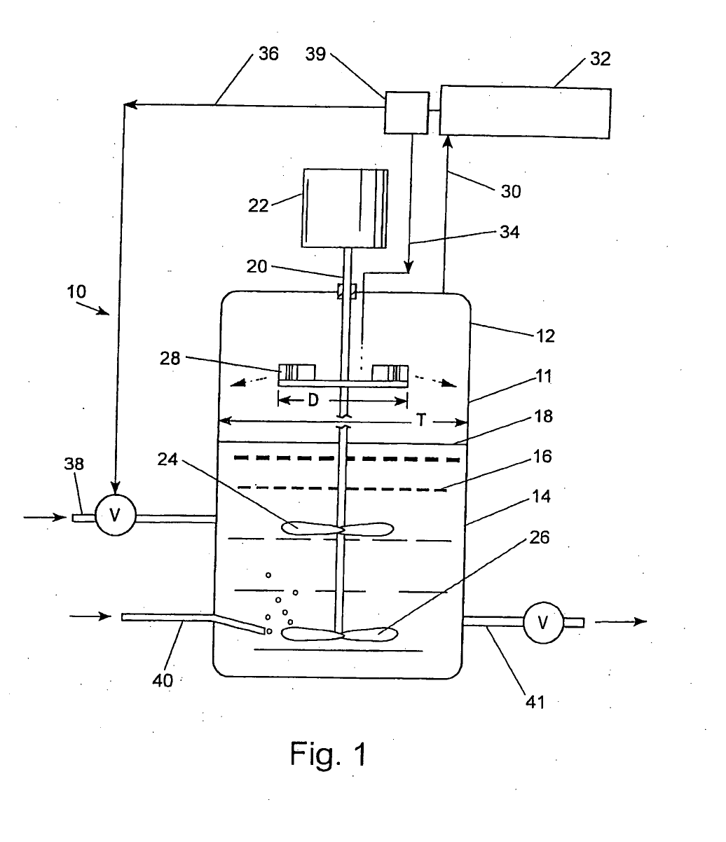

To facilitate further description of several embodiments of the invention, reference is now made to FIG. 1 which is a simplified schematic view of a liquid-gas phase reactor system generally shown at 10. The system 10 includes a vessel 11 having vertically aligned, cylindrical configuration having an inner diameter “T”, an upper section 12 and lower section 14. The vessel 11 is shown including a liquid phase reaction medium 16 which typically comprises a solvent, one or more reactants, and possibly catalysts and other constituents. The liquid phase reaction medium 16 may include suspended solids, dispersions and combinations of immiscible liquids along with dissolved gases. For purposes of FIG. 1, the upper liquid level 18 divides the upper 12 and lower 14 sections of the vessel.

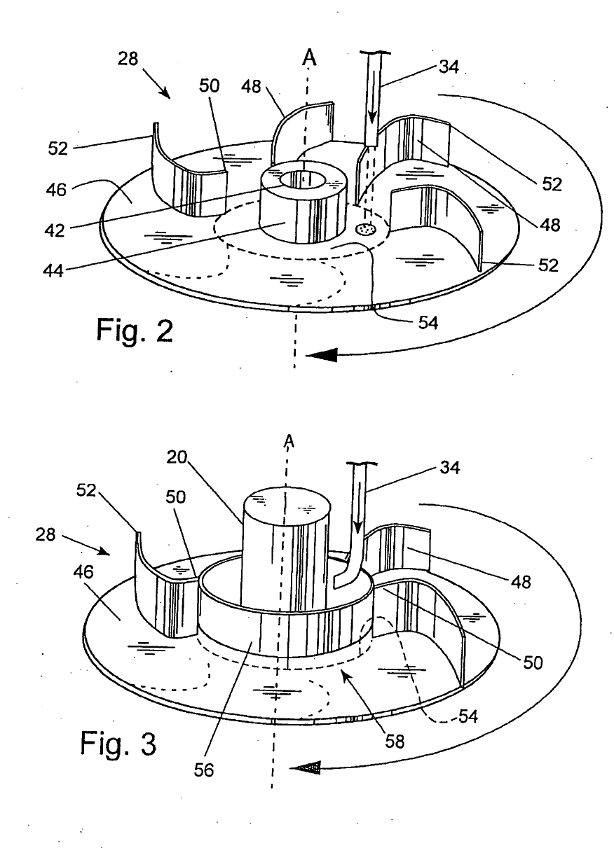

While not necessary for all embodiments of the invention, the reactor system of FIG. 1 includes an agitation device comprising drive shaft 20 extending along an axis of the vessel 11 from the upper section 12 to the lower section 14. The axis is preferably positioned vertically and at a central location within the vessel. The drive shaft 20 may be powered by a conventional motor 22 located outside the vessel 11. The drive shaft 20 is typically cylindrical with a circular cross-section but other configurations, e.g. polygonal, elliptical, etc. may also be used. The agitation device includes upper 24 and lower 26 impeller(s) secured to the drive shaft 20 in the lower section 14 of the vessel 11. Although two impellers are shown, one, two or more impellers are commonly used and are applicable to the invention. Although only shown generically, a variety of specific types of impellers are commonly used in the art and are applicable to various embodiments of the invention. For example, U.S. Pat. No. 6,984,753 describes an agitation device including a combination of an asymmetrical radial impeller and axial impeller, e.g. an upper pitched blade impeller and a lower radial impeller comprising multiple parabolic shaped blades extending radially from a disk with each blade having an upper arc longer than its bottom arc. This type of agitation device operates in downward pumping mode and is applicable to several embodiments of the present invention and is incorporated herein by reference. As will be described in more detail with reference to FIG. 2, the reaction system 10 further includes a slinger 28 having a diameter “D” secured to the drive shaft 20 in the upper section 12 of the vessel 11. Thus, a single drive shaft 20 may operate both the slinger 28 and mixing impellers 24/26.

The vessel 11 includes a vapor outlet 30 in fluid communication with a condenser 32, which in turn is in fluid communication with the vessel 11 via a first 34 and second 36 liquid inlet. The condenser 32 is typically located outside of the vessel 11. The second liquid inlet 36 is shown in fluid communication with a fresh liquid reaction medium inlet 38 at junction valve “V” prior to entering the vessel 11 at a location below the liquid level 18. Although shown including a two liquid inlets 34/36, some embodiments of the invention only require a first liquid inlet 34 from the condenser 32 (or other source of liquid such a fresh liquid feed). Other embodiments have additional inlets including configurations wherein condensate is returned to the vessel via a liquid inlet at a location below the liquid level of the vessel 11, either combined with feed of fresh liquid reaction medium or without. The vapor outlet 30, first 34 and second 36 liquid inlets, fresh liquid reaction medium 38, connecting piping and pressure valves (shown only schematically) and condenser 32 may be selected from those conventionally used in the art, as applied to the specific chemical process. While not shown, the condenser may by combined or associated with other unit operations including solvent strippers, distillation devices and/or other conventional separation devices to condense and separate vapor constituents. In one embodiment, a solvent-rich phase is returned to the vessel whereas a solvent-poor phase is sent to waste treatment. Waste treatment may include additional unit operations including catalyst recovery. Non-condensable constituents may be vented and/or sent to additional unit operations such as scrubbers, incinerators, and gas expanders.

The reactor system may include a condensate control means 39 for controlling the flow of condensate to the vessel. Such fluid control means are well known in the art and may comprise a valve which can be manually controlled or optionally linked to a control mechanism such as a computer for regulating the quantity and direction of flow based upon operating conditions such as internal operating temperature, feed rates, wall fouling, etc. More specifically, condensate may be partitioned by the condensate control means 39 between liquid inlets 34 and 36 based upon the internal temperature of the vessel as measured in the liquid phase reaction medium 16. That is, a higher percentage of the condensate returned (“returned condensate”) to the vessel may be directed to the second liquid inlet 36 in order to dissipate more internal heat; or to the vessel via the first liquid inlet 34 if wall fouling or condensate plugging is detected. In one embodiment, the condensate control means 39 comprises internal sensors positioned throughout the reactor system 10 and linked to a computer (not shown) which controls the flow of condensate from condenser 32 by way of valves (not shown).

A gas inlet 40 distributes gas to desired locations within the vessel 11. While not required in all embodiments of the invention, the gas inlet 40 is commonly used in oxidation reactions and typically delivers oxygen-containing gas, e.g. oxygen, air, oxygen-rich air, etc. to one or more locations near the lower impeller 26. Various configurations are applicable, including multiple gas inlets 40 for introducing gas at multiple locations within the vessel 11. The gas sparger 40 typically includes a remote gas holding tank and pump (not shown) along with inlets to the vessel and discharge nozzles or “spargers” (not shown).

A product outlet 41 is typically located in the lower section 14 of the vessel 11 for removing reaction product effluent from the vessel. Such reaction product effluent often comprises a liquid with some solids content in the form of a slurry, dispersion or emulsion.

FIG. 2 shows a perspective view of one embodiment of the subject slinger 28. The slinger 28 generally comprises a disk-like structure. While shown circular, the slinger may take other shapes, e.g. elliptical, orthogonal, etc, in which case references herein below to the term “radial” will be understood to mean extending from a point near the center to the outer periphery. The term “center” as used in reference to the slinger 28 or upper horizontal surface 46 will be understood to include an area encompassing the axis of rotation, which may differ from the geometric center. The slinger 28 includes a central opening 42 located concentrically about a vertical axis “A” which the drive shaft 20 passes into. A hub 44 or similar means may be used for securing the slinger 28 to the drive shaft 20. Although shown as circular, the central opening 42 may have an alternative shape, e.g. elliptical, orthogonal, etc., but preferably corresponds to the cross-sectional shape of the drive shaft 20. The slinger 28 includes an upper horizontal surface 46. While shown as flat and smooth, the surface may include ridges, channels, or other configurations. While hub 44 is shown positioned on the surface of the upper horizontal surface 46, the hub 44 may be located at alternative positions including below the upper horizontal surface 46. A plurality of vertically raised vanes 48 or “blades” extend radially outward from the center of the upper horizontal surface 46 along a curved path. The curved path of each vane preferably defines a convex arc relative to the direction of rotation (about vertical axis “A”), as specified by the large arrow in FIG. 2. The vanes 48 are preferably thin-walled structures orientated perpendicular with the horizontal surface 46 and have a uniform height “H” and uniform curvature. However, the vanes 48 may vary in height along their length, may vary in height as between vanes, may be tilted or otherwise orientated in a non-perpendicular manner with respect to the upper horizontal surface 46, and may vary in curvature along their length and/or as between individual vanes. The vanes 48 extend radially outward along a curved path from a first end 50 located adjacent the center of the upper horizontal surface 46 to a second end 52 located adjacent the outer periphery of the upper horizontal surface 46. Although the first ends 50 of the vanes 48 may extend directly from the central opening 42 or hub 44, the first ends 50 are preferably spaced therefrom and define the outer periphery of a liquid receiving zone 54 located concentrically about the center of the upper horizontal surface 46. Note that the edge of the first ends 50 of the vane 48 may be perpendicular to the horizontal surface 46 although it need not be. The first liquid inlet 34 is preferably positioned directly above the liquid receiving zone 54 such that condensate, or other liquid being introduced to the vessel 11 is fed onto the liquid receiving zone 54 of the slinger 28. It will be appreciated that multiple liquid inlets may be used to dispense liquid at locations about the liquid receiving zone 54. While shown as a smooth surface, the portion of the upper horizontal surface 46 corresponding to the liquid receiving zone 54 may include a concentric channel or similar structure to facilitate liquid distribution. The liquid receiving zone 54 facilitates the distribution of liquid over the upper horizontal surface 46 and particularly between individual vanes 48.

FIG. 3 illustrates an alternative embodiment of the slinger 28. The embodiment of FIG. 3 shares many common features with the embodiment of FIG. 2 and for the purposes of convenience, similar features have been designated with the same reference numerals. In contrast with FIG. 2, the embodiment of FIG. 3, the liquid inlet 34 is curved near its end such that liquid is directed toward the drive shaft 20. Also, the hub (not shown) is located below the upper horizontal surface 46. In further distinction from the embodiment of FIG. 2, the embodiment of FIG. 3 includes a cup 56 comprising a vertical wall extending upward from a position adjacent the upper horizontal surface 46 and is positioned concentrically about the center of the slinger. The cup 56 includes an open upper section for receiving liquid from the liquid inlet 34, and at least one opening 58 located adjacent to said upper horizontal surface 46 for distributing liquid about the upper horizontal surface 46 of the slinger 28. The cup provides a partial barrier or enclosure about the liquid receiving zone 54. The cup 56 may be secured (e.g. welded) to the first ends 50 of the vanes 48. While the cup 56 has approximately the same height as the vanes 48, in the illustrated embodiment the cup does not extend all the way downward to the upper horizontal surface 46, thus creating an opening 58 adjacent to the upper horizontal surface 46. Thus, liquid introduced into the cup 56 is collected in the liquid receiving zone and radially distributed outward through opening 58 in a relatively uniform manner about the upper horizontal surface 46. In alternative embodiments not shown, the cup may have a height different from than the vanes, and/or may extend downward into contact with the upper horizontal surface 46—in which case the opening 58 may comprise one or more slits, holes or other apertures through the vertical wall of the cup in order to permit liquid to pass radially outward about the upper horizontal surface 46.

As compared with conventional slingers used in liquid-gas phase reactor system, the subject liquid receiving zone 54 distributes more liquid about the majority of the upper horizontal surface 46 of the slinger 28 and results in a more even distribution of liquid between individual vanes 48. In operation, the curved vanes 48 of the slinger 28 provide improved distribution of liquid about the entire cross-sectional area of the vessel 11, thereby reducing wall fouling. Moreover, the curved vanes 48 provide a more homogeneous liquid droplet distribution which improves: i) mixing with the liquid phase reaction medium in the vessel, ii) agglomeration with solids entrained in vapor in the upper section of the vessel 11, and iii) heat and mass transfer with vapor. As the subject slinger is more efficient at distributing liquid about the cross-sectional area of the vessel, less total liquid is necessary for managing wall fouling and/or condensate plugging. Thus, in some embodiments of the invention, a significant portion of liquid introduced to the vessel can be diverted to liquid inlet(s) positioned below the liquid level of the vessel. This aspect of the invention is particularly useful in the oxidation of aromatic alkyls such as xylene (including but not limited to p-xylene, m-xylene, o-xylene and each combination thereof) with solvents such as aqueous acids, e.g. acetic acid, collectively referred to as “liquid reaction medium”. With such reactions, a molecular source of oxygen (e.g. oxygen-containing gas, oxygen peroxide, etc.) is introduced to the liquid reaction medium within a reaction vessel. The resulting reaction is exothermic and the heat generated vaporizes reaction water and solvent which is collected in the upper section of the vessel above the level of the liquid reaction medium. The vapor is condensed and returned to the liquid reaction medium by at least two routes—a slinger located in the upper section of the vessel and a liquid inlet located in the lower section of the vessel below the level of the liquid reaction medium. Such exothermic reactions tend to develop “hot spots” or localized areas of higher temperature within the liquid reaction medium. When equipped with the subject slinger including curved vanes, less than 50% and more preferably less than 30% of the condensate returned to the vessel needs to be returned via the slinger in order to effectively mitigate wall fouling and/or condensate plugging. Consequently, more than 50% and more preferably more than 70% of the returned condensate can be introduced into the liquid reaction medium by way of a liquid inlet located in the lower section of the vessel. As previously described, the introduction of condensate via a liquid inlet located in the lower section of the vessel is more effective at reducing “hot spots” within the liquid reaction medium. Thus, the reaction system can more closely approximate constant chemical potential conditions by optimizing such reaction parameters as temperature, mass gradient, and mass transfer coefficient dependent variables, without significant wall fouling or condensate plugging. Operating under such optimized reactions conditions reduces undesired reactions and consumption of solvent while reducing the total amount of evaporation necessary to maintain desired operating temperatures.

The subject reactor system has been primarily described with reference to preferred embodiments shown in the Figures; however, those skilled in the art will appreciate that a variety of different configurations are also applicable and fall within the scope of the present invention. For example, the general system configuration as described in U.S. Pat. No. 6,984,753 is particularly preferred for oxidation of aromatic alkyls and is incorporated herein by reference; however, different types of agitation impellers, pumping modes (i.e. upward pumping flow vs. downward), gas spargers, draft tubes, etc. are also applicable. Moreover, some embodiments of the invention do not include certain auxiliary equipment such as agitation devices, in which case a drive shaft would preferably only extend to the upper section of the vessel in order to rotate a slinger. Moreover, the drive shaft may not pass through a central opening of the slinger but may be secured via alternative means, e.g. butt-welded to the upper horizontal surface of the slinger. By way of further example, the first liquid inlet 34 may be used to introduce fresh liquid reaction medium rather than condensate. That is, in one embodiment of the invention, the condenser loop (30, 32, 36) is not a required aspect of the invention. In another embodiment, all condensate is returned to the vessel 11 by way of the slinger, with no portion returned via the second liquid inlet 36. In yet another embodiment of the invention, the gas inlet 40 is not included, such as with oxidative reactions utilizing liquid phase oxygen peroxide as a source of molecular oxygen—in which case oxygen peroxide may be introduced via a liquid inlet.

The configuration of a specific liquid-gas phase reactor system will be dependant upon the specific chemical process and scale of operation. However, in general the slinger typically has from 2 to 16 vanes, but preferably 6, 7, 8, 9 or 10 vanes evenly spaced about the upper horizontal surface of the slinger. The slinger is preferably circular with a diameter “D” and the vessel is preferably substantially cylindrical with an inner diameter “T”, wherein D/T is from about 0.05 to 0.7, more preferably about 0.1 to 0.5. The vanes preferably share a uniform vertical height “H” as measured vertically from the upper horizontal surface of the slinger wherein H/D is from about 0.01 to 1. Each vane preferably extends along a curved path of substantially constant curvature having a radius of curvature “R” and an arc length of “L”, wherein the relationship R/D is from about 0.01 to 1000 and L/D is from about 0.01 to 3.14. In a preferred embodiment, R/D, L/D and H/D are the same or different from each other but are independently selected from about 0.1 to 1, but more preferably independently between from about 0.1 to 0.5.

The slingers of the present invention may be fabricated from conventional materials, e.g. steel, titanium, plastic, etc. using conventional fabrication methodologies, e.g. casting, welding, etc. As previously noted, the specific materials of construction will be dictated by the nature of the chemical process, e.g. corrosive environments typically require the use of titanium or stainless steel whereas non-corrosive environments afford the opportunity to use less expensive materials such as carbon based steel. Depending upon the size and configuration of the vessel, the slinger may be constructed in several segments with the various segments being combined within the vessel, such as by bolting or welding segments together. The vanes are preferred secured to the upper horizontal surface of the slinger prior to assemblage of various disk segments within the vessel, such as by way of welding, bolting, use of adhesive, etc. In many industrial scale systems, the slinger will be fabricated from steel with vanes welded to the upper horizontal surface of the slinger, and with various disk segments of the slinger bolted together within the vessel. The slinger is secured to a drive shaft within the vessel by use of bolts and corresponding receiving apertures within a conventional hub.

The subject liquid-gas phase reactor system is useful for conducting a broad range of chemical processes involving both liquid and gas phase constituents within the same vessel. For example, the subject reactor system can be used for fermentations, hydrogenations, phosgenations, neutralizations, chlorinations and oxidation reactions, particularly oxidation of aromatic alkyls.

The gas phase present in the vessel may be added from an external source such as by way of gas spargers, generated as a direct product of reaction, and/or may result from the heat of reaction vaporizing portions of the liquid phase reaction medium. Similarly, the liquid phase present in the vessel may be added from an external source such as by way of a liquid inlet, generated in-situ by condensation, and/or generated as result of the reaction such as the production of reaction water from the oxidation of p-xylene. The reactants for a particular reaction may be introduced to the vessel in liquid phase, gas phase or a combination. The liquid phase typically comprises a reaction medium including a solvent, one or more reactants, catalysts, initiators, and the like.

By way of example, the subject reactor system is particularly well suited for the oxidation of aromatic alkyls. The term “aromatic alkyls” is intended to mean an aromatic ring substituted with one or more alkyl groups each having from one to four carbon atoms, e.g. methyl, ethyl, propyl, isopropyl, and butyl Specific examples include but are not limited to: toluene, p-xylene, m-xylene, o-xylene, and trimethyl benzenes; however, p-xylene is a preferred aromatic alkyl.

Oxidation is preferably accomplished by the addition of a source of molecular oxygen. This is typically accomplished by the introducing an oxygen-containing gas into the liquid reaction medium within the vessel by way of gas spargers. While pure oxygen or high oxygen content air can be used, air is preferred. Other applicable routes include the addition of liquid phase oxygen peroxide into the liquid reaction medium within the vessel by way of a liquid inlet. Those skilled in the art will appreciate that other sources of molecular oxygen may also be use within the context of the present invention.

Preferred oxidation products include aromatic carboxylic acids such as: benzoic acid, orthophthalic acid, isophthalic acid, terephthalic acid (e.g. 1,4-benzenedicarboxylic acid), benzenetricarboxylic acid, trimellitic acid (1,2,4-benzenetricarboxylic acid), 2,6 naphtalene dicarboxylic acid.

The oxidation of aromatic alkyls is typically conducted in an pure or aqueous acid solvent such as benzoic acid or a C₂-C₆ fatty acid, e.g. acetic acid, propionic acid, n-butyric acid, n-valeric acid, trimethylacetic acid, caproic acid and mixtures thereof. A preferred acid solvent is aqueous acetic acid.

The oxidation reaction of aromatic alkyls may be facilitated by the use of catalyst. For example, the oxidation of p-xylene is often catalyzed by a mixture of cobalt and manganese compounds or complexes that are soluble in the selected solvent. Bromide ions are also used as an initiator. Common bromide sources include: tetra bromo ethane, HBr, MeBr, (where “Me” is a metal selected from the alkaline group of metals and/or Co and/or Mn), and NH₄Br.

The oxidation of p-xylene is preferably conducted with air in aqueous acetic acid at a temperature of approximately 180 to 205° C. at approximately 14 to 18 bar.

The invention has been described with respect to many embodiments. However, it should be understood by those skilled in the art that modifications and variations may be made to the invention without departing from the spirit and scope of the invention as defined in the claims.

1. A liquid-gas phase reactor system comprising:
a reaction vessel including an upper and lower section;
a first liquid inlet located in the upper section of said vessel;
a drive shaft extending through at least a portion of the upper section of said vessel;
a slinger secured to said drive shaft and located in the upper section of said vessel below said first liquid inlet, wherein said slinger comprises an upper horizontal surface including a plurality of vertically raised vanes extending radially outward along a curved path.

2. The reactor system according to claim 1 wherein said slinger comprises a liquid receiving zone located about the center of said upper horizontal surface, and wherein said vanes extend radially outward along a curved path from a first end located adjacent the outer periphery of said liquid receiving zone to a second end located adjacent the outer periphery of said slinger.

3. The reactor system according to claim 2 wherein said first liquid inlet is positioned above said liquid receiving zone such that liquid exiting said first liquid inlet is introduced into said liquid receiving zone.

4. The reactor system according to claim 3 wherein said slinger includes a cup comprising a vertical wall positioned concentrically about the center of said upper horizontal surface and enclosing at least a portion of said liquid receiving zone wherein said cup includes at least one opening located adjacent to said upper horizontal surface.

5. The reactor system according to claim 1 wherein said slinger comprises a central opening, wherein said drive shaft extends into said central opening.

6. The reactor system according to claim 1 wherein said upper horizontal surface of said slinger is substantially circular with a diameter (D), said vanes each extend along a curved path of substantially constant curvature having a radius of curvature (R) and an arc length of (L), wherein the relationship R/D is from about 0.01 to 1000, and L/D is from about 0.01 to 3.14.

7. The reactor system according to claim 6 wherein the relationships R/D and L/D may be the same or different from one another, and are each from about 0.1 to 1.

8. The reactor system according to claim 1 wherein said reaction vessel is substantially cylindrical having an inner diameter (T), said upper horizontal surface of said slinger is substantially circular with a diameter (D), and wherein the relationship D/T is from about 0.05 to 0.7.

9. The reactor system according to claim 1 wherein said slinger includes from 2 to 16 vanes each having a vertical height (H) from said upper horizontal surface, and wherein the relationship H/D is from about 0.01 to 1.

10. The reactor system according to claim 1 further comprising:
a second liquid inlet located in the lower section of said reaction vessel;
a vapor outlet located in the upper section of said reaction vessel;
at least one condenser in fluid communication with said vapor outlet, said first liquid inlet and said second liquid inlet; and
a condensate control means for controlling the flow of condensate from said condenser to said reaction vessel by way of said first and second liquid inlets.

11. A liquid-gas phase reactor system comprising:
a reaction vessel having vertically orientated cylindrical inner surface with an inner diameter (T), and an upper and lower section;
a first liquid inlet located in the upper section of said vessel;
a second liquid inlet located in the lower section of said vessel;
a product outlet located in the lower section of said vessel;
a vapor outlet located in the upper section of said vessel;
at least one condenser in fluid communication with said vapor outlet, said first liquid inlet and said second liquid inlet;
a condensate control means for controlling the flow of condensate from said condenser to said vessel by way of said first and second liquid inlets.
a drive shaft extending vertically through said upper and lower sections of said vessel;
at least one mixing impeller secured to said drive shaft and located in the lower section of said vessel;
a slinger comprising a substantially circular upper horizontal surface having a diameter (D), a central opening, a liquid receiving zone located concentrically about said central opening, a plurality of vertically raised vanes extending radially outward along a curved path of substantially uniform height H, constant curvature having a radius of curvature (R) and an arc length of (L) extending from a first end located about the outer periphery of said liquid receiving zone to a second end located adjacent the outer periphery of said slinger;
wherein said drive shaft extends vertically through said central opening and is secured to said slinger in the upper section of said vessel below said first liquid inlet such that liquid exiting said first liquid inlet is introduced into said liquid receiving zone; and
wherein the relationships R/D and L/D may be the same or different from one another and are each from about 0.1 to 1, and
wherein the relationships D/T and H/D may be the same or different from one another and are each from about 0.1 to 0.5.

12. A method for oxidizing of an aromatic alkyl within a liquid-gas phase reactor system comprising the steps of:
providing a vessel having an upper and lower section;
introducing a liquid reaction medium comprising an aromatic alkyl into the vessel;
introducing a source of molecular oxygen to the liquid reaction medium within the vessel;
condensing at least a portion of vapor forming above the liquid reaction medium;
returning at least a portion of the condensate to the liquid reaction medium within the vessel;
wherein more than 50% of the returned condensate is introduced to the liquid reaction medium by way of a liquid inlet located in the lower section of the vessel below the level of liquid reaction medium within the vessel, and less than 50% of the returned condensate is introduced to the liquid reaction medium by way of slinger positioned in the upper section of the reaction vessel above the level of liquid reaction medium within the vessel.

13. The method according to claim 12 wherein more than 70% of the returned condensate is introduced to the liquid reaction medium by way of a liquid inlet located in the lower section of the vessel, and less than 30% of the returned condensate is returned by way of a slinger positioned in the upper section of the reaction vessel above the level of liquid reaction medium within the vessel.

14. The method according to claim 12 wherein the step of returning a portion of the condensate by way of a slinger comprises the step of dispensing condensate upon a slinger which is rotating about a vertical axis, wherein the slinger comprises an upper horizontal surface including a plurality of vertically raised vanes extending outward from the vertical axis along a curved path.

15. The method according to claim 12 wherein the aromatic alkyl comprises a p-xylene and the liquid reaction medium further comprises acetic acid.

16. The method according to claim 13 wherein the aromatic alkyl comprises a p-xylene and the liquid reaction medium further comprises acetic acid.

17. The method according to claim 14 wherein the aromatic alkyl comprises a p-xylene and the liquid reaction medium further comprises acetic acid.
